(19) United States
(12) Patent Application Publication
(10) Pub. No.: US 2026/0305929 A1
(43) Pub. Date: Oct. 8, 2026

(54) URETEROSCOPE DEVICES AND SYSTEMS HAVING A SELECTIVELY EXTENDABLE DISTAL PORTION

(71) Applicant: C.R. BARD, INC., Murray Hill, NJ (US)

(72) Inventors: Justin Wolfe, Lawrenceville, GA (US); Cecille Canary, Atlanta, GA (US)

(21) Appl. No.: 19/100,803

(22) PCT Filed: Aug. 10, 2022
(86) PCT No.: PCT/US2022/039954

§ 371 Date: Feb. 3, 2025

(51) Int. Cl. A61B 1/307; A61B 1/00; A61B 1/005; A61B 1/018

(52) U.S. Cl. CPC A61B 1/307; A61B 1/00042; A61B 1/0008; A61B 1/0016; A61B 1/0052; A61B 1/0057; A61B 1/018; A61B 1/00045

(57) ABSTRACT

An example ureteroscope system (10) including a handpiece (110) and a catheter (104) is described. The handpiece has a distal end region (114), a proximal end region (112), an interior region (155), and a working channel port (118). The catheter extends from the distal end region of the handpiece. The catheter includes a working channel (152) in fluid communication with the working channel port, a proximal portion proximal to the handpiece, and a distal portion (140) distal to the handpiece and selectively extendable to an extended position and selectively retractable to a retracted position relative to the proximal portion. The distal portion includes a distal end defining a working channel opening in fluid communication with the working channel.

### BACKGROUND

Endoscopes of small size are desired in many industrial and medical applications. For example, when natural orifices and lumens of a human body are small, small endoscopes are used for insertion through such orifices and lumens to target locations within the body. For single incision laparoscopy, smaller endoscopes are preferred to provide an inside-the-body view of the surgical site, particularly when the incision itself is of minimal dimensions. Sometimes, patients may feel irritation when an endoscope is being inserted into his or her body, and a smaller endoscope may mitigate such unpleasant experience and may minimize trauma to the patient. Moreover, a physician may improve diagnostic and procedural protocols with a smaller endoscope. For example, transnasal endoscopy may sometimes replace trans-oral endoscopy.

### SUMMARY

Embodiments disclosed herein are related to ureteroscope systems and methods of operating ureteroscope systems. In an embodiment, ureteroscope system includes a handpiece and a catheter. The handpiece has a distal end region, a proximal end region, an interior region, and a working channel port. The catheter extends from the distal end region of the handpiece. The catheter includes a working channel in fluid communication with the working channel port, a proximal portion proximal to the handpiece, and a distal portion distal to the handpiece and selectively extendable to an extended position and selectively retractable to a retracted position relative to the proximal portion. The distal portion includes a distal end defining a working channel opening in fluid communication with the working channel.

In an embodiment, a method of operating a catheter of a ureteroscope in a subject is described. The method includes inserting the catheter of the ureteroscope into a urethra of a subject to a selected position. The method also includes selectively extending a distal portion of the catheter from a retracted position to an extended position using a control member.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A illustrates a ureteroscope as part of an endoscope system having a catheter with an extendable distal portion in a retracted configuration, according to an embodiment.

FIG. 1B illustrates an end view of a distal end of the catheter of the ureteroscope of FIG. 1A, according to an embodiment.

FIG. 1C illustrates the ureteroscope of the endoscope system of FIG. 1A with the extendable distal portion in an extended configuration, according to an embodiment.

FIG. 2 is a side view of the handpiece of the ureteroscope of FIG. 1A with a side of the handpiece removed, according to an embodiment.

FIG. 3 is a side view of the handpiece of a ureteroscope with a side of the handpiece removed, according to an embodiment.

FIG. 4 is a side view of the handpiece of a ureteroscope with a side of the handpiece removed, according to an embodiment.

FIG. 5 is a flow diagram of a method of operating a ureteroscope system, according to an embodiment.

### DETAILED DESCRIPTION

Embodiments disclosed herein relate to devices, systems, and methods of using an endoscope, such as a ureteroscope. At least one, some, or all embodiments of ureteroscope systems and methods described herein include a catheter having a selectively extendable distal portion. For example, at least one, some, or all embodiments of ureteroscope systems and methods described herein include a catheter having a driven or tensioned distal portion. An extendable (e.g. linear deformation) distal portion of the catheter of the ureteroscope according to at least one, some, or all embodiments described herein results in the technical effect of allowing the operator to reach a lower pole calyces of a kidney (e.g., a stone in a lower calyces) of a subject, particularly under severe deflection of the catheter.

At least one, some, or all embodiments of ureteroscope systems described herein include a hydrophilic coating on at least a portion (e.g., the distal portion) of the catheter. The hydrophilic coating on at least a portion of the catheter, and in particular the distal portion of the catheter as the distal portion is translate forward or extends, results in the technical effect of reducing the chance of subject injury (e.g., ureteral/uretheral abrasion) and/or suffering from the catheter. The hydrophilic coating results in the technical effect of facilitating easier insertion and/or movement of the catheter into the anatomy of the subject. In some embodiments, at least a portion of the catheter may be dipped into a hydrophilic material to coat at least a portion of the catheter with a hydrophilic material (e.g., carbopol coating).

In some embodiments, the ureteroscope may include a small, plastic gripper that goes over at least some of the catheter. The gripper results in the technical effect of allowing the medical provider to advance the catheter without having to touch the hydrophilic coating of the catheter. This gripper may be left in place once the catheter is advanced to the desired location, or may have a slit that allows the medical provider to remove the gripper after insertion.

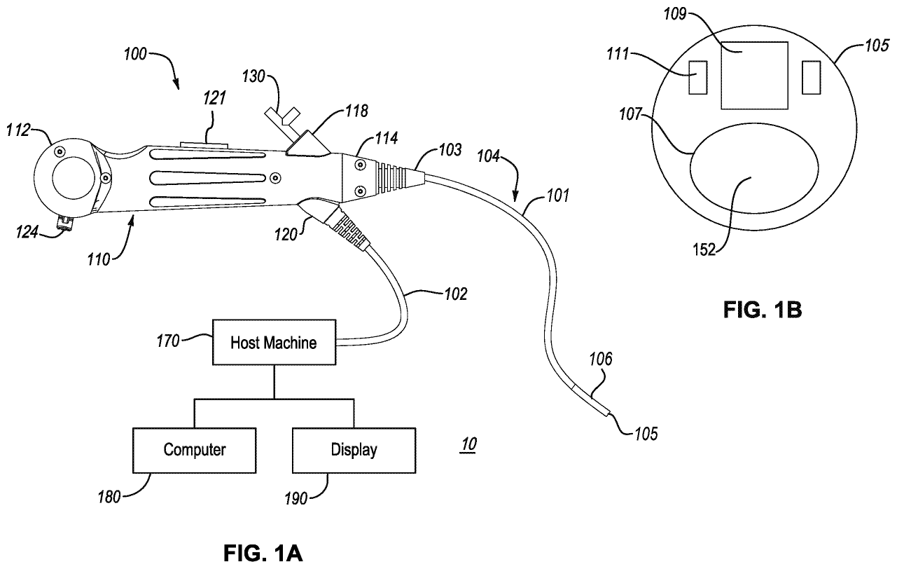

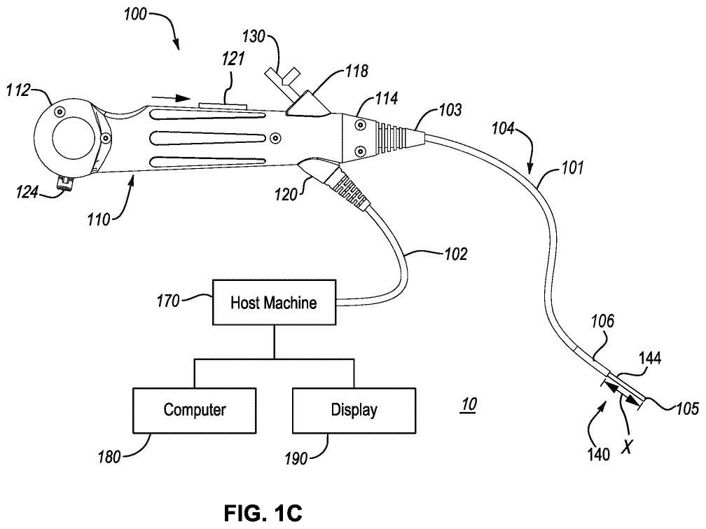

FIG. 1A illustrates an endoscopic system 10. In an embodiment, the endoscopic system 10 includes a digital ureteroscope 100 operably coupled to one or more electronic devices, such as a host machine 170 and one or more external terminals including a display 190 and a computer 180. The ureteroscope 100 may be operably coupled to one or more of the host machine 170, the computer 180, or the display 190 wirelessly or through a cable 102. The ureteroscope 100 includes a catheter 104 and a handpiece 110 having a catheter end 114 (e.g., distal end region) and a control end 112 (e.g., proximal end region) opposite to the catheter end 114. As the control end 112 is opposite to the catheter end 114, the control end 112 and the catheter end 114 are at different ends of the handpiece 110 and may face different directions relative to one another. In an embodiment, both the catheter 104 and the handpiece 110 are disposable. In some embodiments, the catheter 104 and the handpiece 110 are manufactured as an integral part, or the catheter 104 is fixed with the handpiece 110 via a handpiece-catheter connector 103. Alternatively, only the catheter 104 is disposable, and the handpiece 110 may be sterilized and reused multiple times. In this case, the catheter 104 is removably connected to the handpiece 110 and the catheter end 114 via a handpiece-catheter connector 103.

The catheter 104 of the ureteroscope 100 may be used for imaging an interior surface of a tubular structure, such as a lumen in the body of human or animal. For example, the catheter 104 may be inserted via a subject's urethra to access various parts of the urinary tract. The ureteroscope 100 also may be used for imaging, examination, and/or treatment within other organs of the body, such as a kidney. As provided above, at least one, some, or all embodiments of the ureteroscope 100 described herein provide the technical effect that results in improved treatment of calyces of a kidney. However, it should be appreciated that the ureteroscope 100 may be employed as an industrial endoscope when tubular structure is a part of an industrial apparatus, an equipment, a product, a machine, a production line, and the like. In some embodiments, the catheter 104 may serve as a tether, and may include a plurality of scale markings or fiducials that enable a physician to measure a distance traveled by optoelectronic module into the tubular structure, such as a lumen of a body.

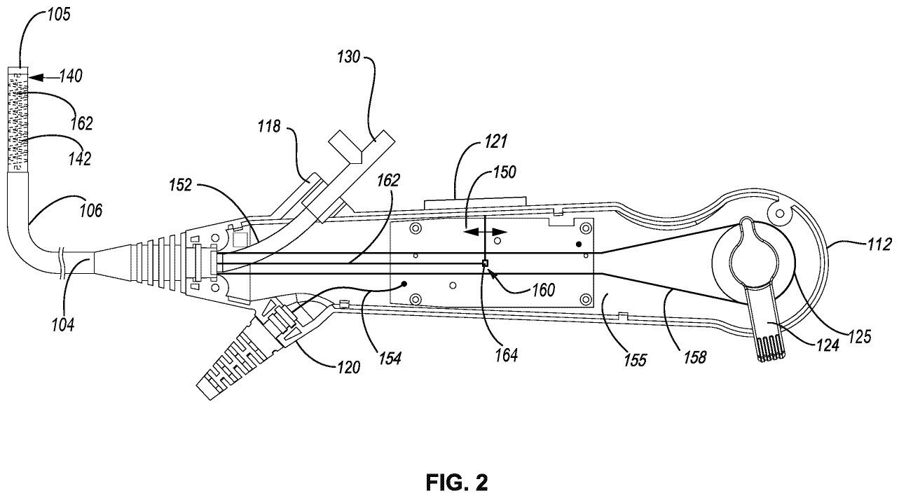

Other structure(s) may be built into the ureteroscope 100 as desired. For example, the handpiece 110 may include a steering controller 124 configured to control one or more steering wires 158 (FIG. 2) that are connected to an active bend portion 106 of the catheter 104 to deflect the distal end 105 to the desired location. Accordingly, a user may bend or curve the catheter 102 by moving the steering controller 124 on the handpiece 110. In some embodiments, a wire wheel 125 is rotatably secured to the interior region 155 of the handpiece 110. The steering controller 124 on the handpiece 110 may be directly or indirectly connected to the wire wheel 124 and configured to at least partially and selectively rotate the wire wheel 125 in two different directions. The one or more steering wires 158 are secured to the active bend portion 106 of the catheter 105 and adjustable responsive to rotation of the wire wheel effective to at least partially bend the active bend portion 106 one or more (e.g., at least two) directions. In some embodiments, the active bend portion 106 of the catheter 104 is made of certain flexible medical-grade polymer materials to allow for steerability of the catheter 104 and reduce discomfort to patients. The catheter 104 may include the active bend portion 106 and a passive bend portion 101. The active bend portion 106 may be generally distal to the handpiece 110 and may be controlled by one or more (e.g., two) control wires 158 (e.g., steel wires) to bend in two directions, one being used for bending the active bend portion 106 to one direction with a 0° to about 275° range, the other being used for bending the active bend portion 106 to the opposite direction with the same degree range. Other number of steering wires (e.g., 1, 3, or 4) may be implemented here as well. In contrast, the passive bend portion 101 may be between the active bend portion 106 and the handpiece 110 and may bend according to the shape of urinary tract during insertion. Suitable active bend portions of catheters are disclosed in U.S. Pat. No. 11,129,519 and U.S. Patent Publication No. 2018/0140177, the disclosures of which are incorporated herein, in their entirety, by this reference.

In at least one, some, or all embodiments of the ureteroscope 100, the catheter 104 having a selectively extendable and/or retractable distal portion 140 (visible in FIG. 1C). The distal portion 140 is selectively adjustable to at least extend from a retracted configuration (e.g., retracted position, shown in FIG. 1A) to an extended configuration (e.g., extended position, shown in FIG. 1C). In some embodiments, the distal portion 140 is also selectively adjustable to retract from the extended configuration to the retracted configuration. An extendable (e.g. linear deformation) distal portion 140 of the catheter 104 of the ureteroscope 100 according to at least one, some, or all embodiments described herein results in the technical effect of allowing the operator to reach a lower pole calyces of a kidney (e.g., a stone in a lower calyces) of a subject, particularly under severe deflection of the catheter 104. In some embodiments, the catheter 104 includes a proximal portion (e.g., at least some of the passive bend portion 101) proximal to the handpiece 110.

As shown in FIG. 1C, the distal portion 140 of the catheter 104 is distal to the handpiece 110 and may extend from a region of the catheter 104 generally distal to the handpiece 110. In some embodiments, the distal portion 140 of the catheter extends from an end region of the active bend portion 106 such that, when the distal portion 104 is extended, the active bend portion 106 is disposed between the distal portion 140 and the passive bend portion 101 of the catheter 104. This configuration of the catheter 104 results in the technical effect of allowing an operator to bend the end of the catheter 104 a desired direction, and if necessary, linearly extend the distal end 105 of the catheter 104 to position the distal end 105 of the catheter 104 closer to a target or area in need of treatment or viewing (e.g., calyces of a kidney). In some embodiments, the distal portion 140 is configured to linearly extend from the active bend portion 106 (e.g., lengthen the catheter 104 as a whole or extend relative to the retracted configuration) a distance X of about 2 mm to about 30 mm, about 2 mm to about 15 mm, about 15 mm to about 30 mm, about 2 mm to about 10 mm, about 10 mm to about 20 mm, about 20 mm to about 30 mm, about 2 mm to about 5 mm, about 5 mm to about 10 mm, about 10 mm to about 15 mm, about 15 mm to about 20 mm, about 20 mm to about 25 mm, or about 25 mm to 30 mm. In some embodiments, the operator of the ureteroscope 100 can selectively extend the distal portion 140 to a preferred or selected distance X based on the circumstances during use. For example, the distal portion 140 may be configured to selectively extend from the active bend portion 106 (or lengthen the catheter 104 as a whole) to a selected position within any of the ranges provided above.

The ureteroscope 100 includes an actuator and a control member configured to allow an operator of the ureteroscope to selectively extend and/or retract the distal portion 140, according to an embodiment. For example, turning to FIG. 2, the ureteroscope 100 may include an actuator 160 disposed in the interior region 155 of the handpiece 110 and operably connected to the distal portion 140 and configured to at least extend and/or retract the distal portion 140 of the catheter 104 relative to the proximal portion of the catheter 104. More particularly, the actuator 160 may include a base member 164 and one or more wires 162 or cables secured to the base and the distal portion 140 of the catheter 104. The base member 160 may be positioned in the interior region 155 of the handpiece 110 and selectively moveable (e.g., linearly) in the interior region 155 between a first position and a second position. The distal portion 140 of the catheter 104 may be tensioned by the one or more wires 162 when the base is in the first position such that the distal portion is held in the retracted position when the base member 164 is in the first position. Moving the base member 162 to the second position in the interior region 155 of the handpiece 162 may extend the distal portion 140 to the extended position.

The distal portion 140 is operably coupled to the actuator 160 such that the distal portion 140 selectively extends and/retracts responsive to the actuator 160. For example, in some embodiments, the distal portion 140 of the catheter 104 includes a coiled spring 142 or bellows housed at least partially therein and having a distal end region secured directly or indirectly to the one or more wires or cables 162. In some embodiments, the distal end may include any of a variety of springs or other biasing members, such as a compression spring, a tension spring, a coiled spring, a torsion spring, a spring belt, a leaf spring, and/or a compliant piece of material. In some embodiments, the distal portion 140 includes a wall or end piece proximate the distal end 105 having the one or more wires 162 or cables secured thereto and holding the coiled spring 142 or bellows in the distal portion 140. In some embodiments, the wall or end piece may have the optoelectronic module 109 and/or the light source 111 embedded therein or secured thereto opposite to the coiled spring 142. When the one or more cables 162 are holding the coiled spring 142 or bellows at least partially in tension, the distal portion may be held in the retracted position. Moving the base member 164 to the second position in the interior region 155 of the handpiece 110 may at least partially release tension in the coiled spring 142 or bellows such that the coiled spring elongates and pushes the distal end 105 outward linearly to extend the distal portion 140 to the extended configuration. Returning the base member 164 to the first position in the interior region 155 of the handpiece 110 may tension the one or more wires 162 or cables and pull the coiled spring 142 or bellows inward toward the handpiece 110, thereby linearly shortening the coiled spring 142 or bellows and returning the distal portion 140 at least partially toward the retracted configuration. In some embodiments, the distal end 140 may include a telescoping distal end that extends and/or retracts responsive to the actuator 160, and the coil spring 142 may be absent.

In some embodiments, the ureteroscope 100 includes a control member 121 operably connected to the actuator 160 to selectively extend and/or retract the distal portion of the catheter 104 relative to the proximal portion of the catheter 104. For example, the control member 121 may include a sliding tab, grip, or button on the exterior of the handpiece and secured to the base member 164. In some embodiments, the base member includes a rigid arm extend from the sliding tab, grip, or button into the interior region 155 of the handpiece. The handpiece 110 may include an elongated slot that allows the sliding tab, grip, or button to slide from a first position corresponding to the first position of the base member 164 to a second position corresponding to the second position of the base member 164. In some embodiments, the handpiece 110 includes one or more locking elements between the first position and the second position of the sliding tab, grip, or button that allow an operator to lock the sliding tab, grip, or button in a selected position between the first position and the second position of the sliding tab, grip, or button, thereby holding the distal portion at a selected partially extended configuration. The locking elements may include addition perpendicular slots or a friction locking element. In some embodiments (not shown), the control member 121 is separate from the handpiece 110. For example, the control member 121 may include an electronic device remote or separated from the handpiece, but operably connected to the communication interface 150 and/or the base 164.

The ureteroscope 100 may include an optoelectronic module (e.g., a camera or other imager) for imaging the interior of the subject. For example, turning to FIG. 1B, an optoelectronic module 109 (e.g., image sensor) and at least one light source 111 may be located in a distal end 105 of the catheter 104 or other location in the catheter 104. The optoelectronic module 109 may include a micro camera module having an image sensor microchip, a set of micro lenses, and a micro illumination module. Suitable optoelectronic modules are disclosed in U.S. Pat. No. 9,942,452, which is incorporated herein, in its entirety, by this reference. In some embodiments, the optoelectronic module 109 may be positioned in a rigid or semi-rigid shell-like housing at the distal end 105 configured for insertion into the tubular structure for imaging its interior surface. For example, the optoelectronic module 109 may be inserted into a patient's body through a natural body orifice, such as the mouth, nose, urethra, bladder, vagina, or anus. The ureteroscope 100 may therefore have different configurations for use as a gastrointestinal, a colonoscope, endoscopic ultrasound (EUS), endoscopic retrograde cholangiopancreatography (ERCP), or other suitable application. Applications of the ureteroscope 100 include diagnostic observation associated with endometrial polyps, infertility, abnormal bleeding, and pelvic pain, and surgical procedure such as embryo growth arrest and uterine malformation etc.

The ureteroscope 100 may include a receiving device or communication interface 150 (FIG. 2) generally located outside the catheter 104 for receiving the signal from an image sensor within the optoelectronic module 109. For example, the catheter 104 may include at least one electrical lead 162 (FIG. 2) that is coupled to the optoelectronic module 109 and conveys an electrical signal from optoelectronic module 109 to the communication interface 150. The communication interface 150 may be positioned within an interior region 155 (FIG. 2) of the handpiece 110 or adjacent to the handpiece 110.

The catheter 104 may be configured to couple the optoelectronic module 109 to the circuitry within the handpiece 110 in any suitable manner. For example, the availability of low-cost modular imaging system components enables the manufacture of a disposable components of the ureteroscope 100 at very low cost. In an embodiment, the catheter 104 is configured to detachably couple the optoelectronic module 109 to circuitry within the handpiece 110. In this manner, the catheter 104 and the optoelectronic module 109 are disposable, and may be detached from the handpiece 110 after a single patient use, thus eliminating the need for sterilization or reprocessing and reducing contamination risks. The handpiece 110 may be disinfected for subsequent reuse with a catheter 104 and optoelectronic module 109 for a different patient.

Returning to FIG. 1A, the endoscopic system 10 may include one or more electronic devices for processing and displaying the image data received from the optoelectronic module 109 of the ureteroscope 100. For example, the endoscopic system 10 may include one or more of a host machine 170 having a microprocessor, a computer 180 having a microprocessor, and a display 190. The host machine 170 may be connected to one or more terminals of the computer 180 and the display 190 for further processing and displaying the image data from the optoelectronic module 109. The host machine 170 or the computer 180 may be programmed with image processing software that takes as input the image data output from the optoelectronic module 109 of the ureteroscope 100 and generates two-or three-dimensional reconstructions of the body lumen that may be displayed on the display 190. Accordingly, a processor in at least one of the host machine 170, the computer 180, or the display 190 may be programmed with software that accepts as input a plurality of still images of an object generated by the optoelectronic module 109, and then output for display a three-dimensional rendering of the object based on the plurality of still images. The display 190 may include any suitable display, and may be configured to display a moving image (e.g., movie or video) or a still image collected by the image sensor of the optoelectronic module 109. Although shown in FIG. 1A as separate blocks, the host machine 170, the computer 180, and the display 190 may include a single device, two devices, three devices, or more than three devices.

The endoscopic system also may include a cable 102 configured to operably couple the ureteroscope to at least one of the host machine 170, the computer 180, the display 190, and/or other electronic devices (such as a remote control member 121). The cable 102 may electrically couple the retrieving device 150 (FIG. 2) of the ureteroscope 100 to at least one of the host machine 170, the computer 180, or the display 190. The cable 102 also may allow the communication interface 150 to communicate with and receive electric power from the host machine 170 or other power sources. The cable 102 also may be configured to allow the communication interface 150 to transmit image data captured at the optoelectronic module 109 to the host machine 170 for processing, storing, and displaying.

Turning to FIG. 2, which illustrates a side view of the handpiece 110 with a portion of the handpiece 110, the ureteroscope 100 may include a communication interface 150 or host interface housed in the handpiece 110. The communication interface 150 may contain, for example, one or more of a processor board, a camera board and frame grabber, or a power source. The processor board may be coupled by the cable 102 to the host machine 170 for storage and retrieval of images generated by ureteroscope 100. The communication interface 150 also may be configured to communicate with and receive electric power from the host machine 170 or other power source via the cable 102. The communication interface 150 also may transmit image data captured at the distal end 105 to the host machine 170 for processing, storing, and displaying. The communication interface 150 may be connected to the cable 102 through one or more wires 154 and/or connected to the optoelectronic module 109 through one or more electrical leads 162.

Alternatively or in addition, the communication interface 150 or handpiece 110 may include an antenna and a wireless chipset, e.g., compliant with the IEEE 802.11 Wi-Fi standards, for wirelessly transmitting the video or still image generated by the ureteroscope 100 to the host machine 170, the computer 180, or the display 190 without the cable 102. For example, the communication interface 150 may include a wireless interface configured to implement various protocols, including but not limited to Wi-Fi, Bluetooth, ZigBee, Z-Wave, etc. This arrangement may be useful in a physician's office because it permits the computer and display to be placed outside of the sterile field, while also allowing the physician greater maneuverability during use of the ureteroscope 100. In other embodiments, the communication interface 150 may be omitted and the image data may be transmitted directly to the host machine 170 and/or the computer 180.

The ureteroscope 100 may be operated to perform or complete selected tasks manually, automatically, or a combination thereof. Some ureteroscopic functions may be implemented with the use of components that comprise hardware, software, firmware or combinations thereof. While general-purpose components such as general purpose computers or oscilloscopes may be used in the ureteroscope 100, dedicated or custom components such as circuits, integrated circuits or software may be too. For example, some functions are implemented with a plurality of software instructions executed by one or more data processors, which is part of a general-purpose or custom computer. The one or more data processors may be in at least one of the communication interface 150, the host machine 170, the computer 180, or the display 190. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more input devices (e.g., allowing input of commands and/or parameters) and output devices (e.g., allowing reporting parameters of operation and results).

The handpiece 110 also includes a working channel port 118 and a working channel opening 107 (FIG. 1B) that provides fluid communication between the working channel port 118 and the distal end 105 of the catheter 104. The handpiece 110 also may include a working channel connector 130 that is connected or attached to the working channel port 118 and a working channel 152 that provides fluid communication between working channel port 118 and the working channel opening 107 through the handpiece 110 and/or the catheter 104. The working channel 152 may extend through the distal portion 140 to include the working channel opening 107 at the distal end 105 of the catheter 104. The working channel 152 may include a single, continuous material through the handpiece 110 and the catheter 104. In some embodiments, the working channel 152 includes a first portion in the interior region 155 of the handpiece 110 and a second portion in the catheter 104. The first portion and the second portion of the catheter 152 may include different materials or configurations that are fluidly connected to one another. At least one of the working channel port 118 or the working channel connector 130 is configured to engage with various surgical instruments and irrigation devices such as a laser fiber, as needed, for operations such as stone breaking and retrieval, etc. The working channel port 118 may be positioned on a lower/bottom or an upper/top portion of the handpiece 110, proximate to the catheter end 114 of the handpiece 110. The working channel port 118 may be positioned less than one-half of a distance from the catheter end 114 to the control end 112, less than one-third of the distance from the catheter end 114 to the control end 112, less than one-quarter of the distance from the catheter end 114 to the control end 112, or less than one-fifth of the distance from the catheter end 114 to control end 112.

The handpiece 110 also may include a cable port 120 for receiving or connecting the cable 102 (FIG. 1A), thereby operably coupling the ureteroscope 100 to at least one of the host machine 170, the computer 180, or the display 190. Both the cable port 120 and the working channel port 118 also may be positioned on a lower or bottom portion of the handpiece 110, proximate to the catheter end 114 of the handpiece 110. For example, both the cable port 120 and the working channel port 118 may be positioned less than one-half of a distance from the catheter end 114 to the control end 112, less than one-third of the distance from the catheter end 114 to the control end 112, less than one-quarter of the distance from the catheter end 114 to the control end 112, or less than one-fifth of the distance from the catheter end 114 to control end 112. Alternatively, the handpiece 110 may further include a compact battery module for supplying power to the optoelectronic module 109 and the at least one light source 111. The power source in the handpiece 110 may be, for example, one or more conventional dry-cell disposable batteries or lithium ion rechargeable batteries.

Moreover, while the ureteroscope 100 shown in FIGS. 1-2 includes a cable port 120 positioned proximate to the catheter end 114 and distal to the control end 112, in some embodiments, the cable port 120 is absent from the handpiece 110 or positioned proximate to the control end 112 and distal to the catheter end 114. Accordingly, in some embodiments, the cable port 120 is absent from the handpiece 110 or positioned proximate to the control end 112. Furthermore, while the ureteroscope 100 shown in FIGS. 1-2 includes a working channel port 118 disposed on the rounded first surface 126 along the longitudinal central plane proximate to the catheter end 114 and distal to the control end 112, in some embodiments, the working channel port 118 may be disposed elsewhere on the handpiece 110. Accordingly, in some embodiments, the working channel port 118 may be disposed elsewhere than along the longitudinal central plane proximate to the catheter end 114.

In at least one, some, or all embodiments of the ureteroscope 100, at least a portion of the catheter 104 may be a lubrisciously coated catheter, such as a hydrophilic coating disposed thereon. For example, at least the distal portion 140 may include the hydrophilic coating. In some embodiments, at least one (e.g., both) of the distal portion 140 and/or the active bend portion 106 include the hydrophilic coating disposed thereon. In some embodiments, all of the catheter 104, including the distal portion 140 and the active bend portion 106, include the hydrophilic coating disposed thereon. The hydrophilic coating on the catheter 104 results in the technical effect of allowing for easier insertion of the catheter 104 into the subject and less ureteral trauma/tearing during insertion. The hydrophilic coating may include a carbopol or polyvinylpyrrolidone (PVP), coating.

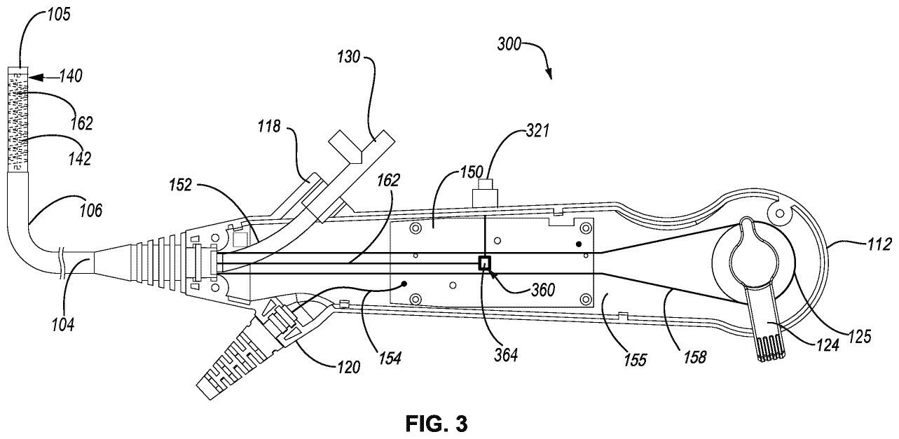

Turning now to FIG. 3, in some embodiments, an actuator 360 of a ureteroscope 300 may include a motor 364 positioned in the interior region 155 of the handpiece 110. Unless otherwise noted, the ureteroscope 300 may include any aspect of the ureteroscope 100, including the catheter 104, the active bend portion 106, the selectively extendable and/or retractable distal portion 140, the coiled spring 142, the one or more wires or cables 162, the optoelectronic module 109, and/or the light source 111. FIG. 3 is a side view of the handpiece 110 of the ureteroscope 300 with a side of the handpiece 110 removed, according to an embodiment.

The motor 364 may be operably connected to the distal portion 140 and configured to extend and retract the distal portion 140 of the catheter 104 relative to the proximal portion of the catheter 104. For example, the one or more wires 162 or cables may be secured to the motor 364 and the distal portion 140 of the catheter 104. The motor 364 may be configured to increase (e.g., unwind or release) an amount of the one or more wires 162 or cables between the motor 364 and the distal portion 140 of the catheter 104 to extend the distal portion 140 to the extended position. The motor 364 also may be configured to decrease (e.g., wind or retract) an amount of the one or more wires 162 or cables between the motor 364 and the distal portion 140 of the catheter 104 to retract the distal portion 140 to the retracted position. When the one or more cables 162 are holding the coiled spring 142 at least partially in tension, the distal portion 140 may be held in the retracted position. When the motor 364 increases (e.g., unwinds or releases) an amount of the one or more wires 162 or cables between the motor 364 and the distal portion 140 of the catheter 104, the tension in the coiled spring 142 may be at least partially released such that the coiled spring elongates and pushes the distal end 105 outward linearly to extend the distal portion 140 to the extended configuration. When the motor 364 decreases (e.g., winds or retracts) an amount of the one or more wires 162 or cables between the motor 364 and the distal portion 140 of the catheter 104, tension in the one or more wires 162 or cables may pull the coiled spring inward toward the handpiece 110, thereby linearly shortening the coiled spring 142 and returning the distal portion 140 at least partially toward the retracted configuration.

In some embodiments, the distal portion 140 telescopes at least partially into the catheter in the retracted position. In some embodiments, the motor 364 is positioned in the handpiece 140 proximate to the catheter 104, and the one or more wires 162 or cable are absent. Instead, the catheter 104 may include an outer tube secured to the handpiece 110 and an inner tube secured to the motor 364 and including the distal end 105 of the catheter. The inner tube may be slidable or movable within the outer tube, and the inner tube may be secured to the motor 364 such that the motor 364 may push, force, or direct a portion of the inner tube out of the distal end of the outer tube to linearly extend the catheter 104. The motor 364 also may pull or retract the inner tube to retract the inner tube back into the outer tube, thereby linearly retracting the catheter 104.

In some embodiments, the ureteroscope 300 includes a control member 321 operably connected to the actuator 360 to selectively extend and/or retract the distal portion 140 of the catheter 104 relative to the proximal portion of the catheter 104. For example, the control member 321 may include a button, sliding tab, or grip, on the exterior of the handpiece and operably coupled to the motor 364. In some embodiments (not shown), the control member 321 is separate from the handpiece 110. For example, the control member 321 may include an electronic device remote or separated from the handpiece 110, but operably connected to the communication interface 150 and/or the motor 364.

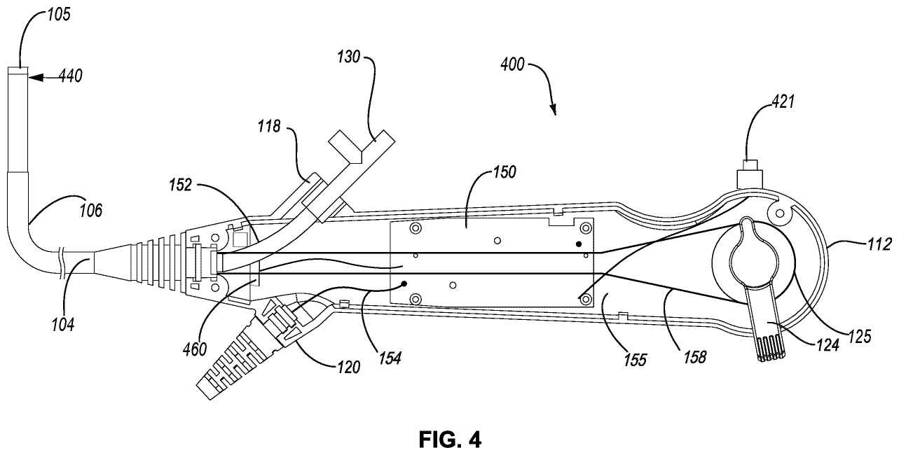

Turning now to FIG. 4, in some embodiments, an actuator of a ureteroscope 400 may include a pneumatic or hydraulic pump 460 positioned in the interior region of the housing 1110. Unless otherwise noted, the ureteroscope 400 may include any aspect of the ureteroscope 100, including the catheter 104, the active bend portion 106, the optoelectronic module 109, and/or the light source 111. FIG. 4 is a side view of the handpiece 110 of the ureteroscope 400 with a side of the handpiece 110 removed, according to an embodiment.

The catheter 104 of the ureteroscope 400 also includes a selectively extendable and/or retractable distal portion 440. The pump 460 may be positioned in the handpiece at least proximate (e.g., adjacent) to the catheter 104. The pump 460 may selectively pneumatically or hydraulically increase a pressure in at least a portion of the catheter 104 effective to deform at least the distal portion 440 of the catheter 104 to extend the distal portion 440 of the catheter 104 to the extended position. The pump 460 also may selectively pneumatically or hydraulically decrease a pressure in at least a portion of the catheter 104 effective to deform at least the distal portion 440 of the catheter to retract the distal portion 440 of the catheter 104 to the retracted position. For example, the distal portion 440 may include a bellows that extends with increased pressure from the pump 460, and retracts when the pressure is not longer exerted by the pump 460.

In some embodiments, the ureteroscope 400 includes a control member 421 operably connected to the pump 460 to selectively extend and/or retract the distal portion 440 of the catheter 104 relative to the proximal portion of the catheter 104. For example, the control member 440 may include a button, sliding tab, or grip, on the exterior of the handpiece and operably coupled to the pump 460. In some embodiments (not shown), the control member 421 is separate from the handpiece 110. For example, the control member 421 may include an electronic device remote or separated from the handpiece 110, but operably connected to the communication interface 150 and/or the pump 460.

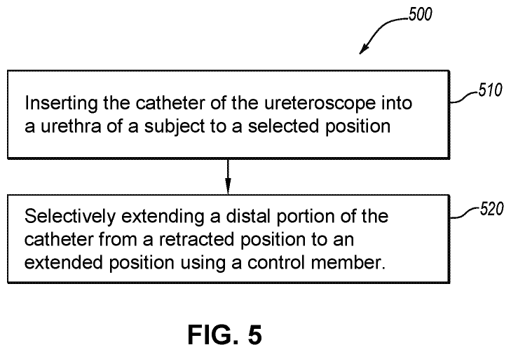

FIG. 5 is a flow diagram of a method 500 operating a catheter of a ureteroscope in a subject, according to an embodiment. The method 500 may include an act 510 of inserting the catheter of the ureteroscope into a urethra of a subject to a selected position. The method 500 also may include an act 520 of selectively extending a distal portion of the catheter from a retracted position to an extended position using a control member.

In some embodiments, the act 520 may include activating, with the control member, an actuator disposed in the interior region of the handpiece and operably connected to the distal portion to extend the distal portion of the catheter relative to the proximal portion of the catheter. More particularly, in some embodiments, activating, with the control member, an actuator disposed in the interior region of the handpiece and operably connected to the distal portion to extend the distal portion of the catheter relative to the proximal portion of the catheter may include moving a base of the actuator positioned in the interior region of the handpiece from a first position to a second position to at least partially release tension on one or more wires or cables secured to the base and the distal portion of the catheter. The distal portion of the catheter may include a coiled spring or bellow housed at least partially therein and having a distal end region secured to the one or more wires or cables such that moving the base of the actuator from the first position to the second position at least partially releases tension on one or more wires or cables and the coiled spring in the distal portion. In some embodiments, activating, with the control member, an actuator disposed in the interior region of the handpiece and operably connected to the distal portion to extend the distal portion of the catheter relative to the proximal portion of the catheter may include activating a motor positioned in the interior region of the handpiece to increase an amount of one or more wires or cables between the motor and the distal portion of the catheter to extend the distal portion to the extended position. In some embodiments, activating, with the control member, an actuator disposed in the interior region of the handpiece and operably connected to the distal portion to extend the distal portion of the catheter relative to the proximal portion of the catheter may include activating a pneumatic or hydraulic pump positioned in the interior region of the housing to pneumatically or hydraulically increase a pressure in at least a portion of the catheter and deform at least the distal portion of the catheter to extend the distal portion of the catheter to the extended position.

In some embodiments of the method 500, the distal portion telescopes out from the catheter when the distal portion is selectively extended, the control member may include a selectively slidable tab on the handpiece, the control member includes an electronic device separate from the handpiece, and/or at least a distal portion of the catheter includes a hydrophilic coating on at least the distal portion of the catheter. In some embodiments, the method 500 also includes an act of bending an active bend portion of the catheter before selectively extending the distal portion of the catheter. In some embodiments, selectively extending the distal portion of the catheter includes selectively extending the distal portion of the catheter about 2 mm to about 30 mm.

The acts of the method 500 described above, including but not limiting to the acts 510 and 520, are for illustrative purposes. For example, the acts of the method 500 may be performed in different orders, split into multiple acts, modified, supplemented, or combined. In an embodiment, one or more of the acts of the method 500 may be omitted from the method 500. Any of the acts of the method 500 may include using any of the handpieces 110, ureteroscopes 100, 300, 400 and/or the system 10 disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.” While various aspects and embodiments have been disclosed herein, other aspects and embodiments are contemplated. The various aspects and embodiment disclosed herein are for purposes of illustration and are not intended to be limiting.

What is claimed is:

1. A ureteroscope system, comprising:
a handpiece having a distal end region, a proximal end region, an interior region, and a working channel port; and
a catheter extending from the distal end region of the handpiece, the catheter including a working channel in fluid communication with the working channel port, a proximal portion proximal to the handpiece, and a distal portion distal to the handpiece and selectively extendable to an extended position and selectively retractable to a retracted position relative to the proximal portion, the distal portion including a distal end defining a working channel opening in fluid communication with the working channel.

2. The ureteroscope system of claim 1, further comprising:
an actuator disposed in the interior region of the handpiece and operably connected to the distal portion and configured to extend and retract the distal portion of the catheter relative to the proximal portion of the catheter; and
a control member operably connected to the actuator to selectively extend and retract the distal portion of the catheter relative to the proximal portion of the catheter.

3. The ureteroscope system of claim 2, wherein the actuator includes:
a base positioned in the interior region of the handpiece and moveable in the interior region between a first position and a second position;
one or more wires or cables secured to the base and the distal portion of the catheter;
wherein the distal portion of the catheter is tensioned by the one or more wires when the base is in the first position such that the distal portion is held in the retracted position when the base is in the first position and moving the base to the second position extends the distal portion to the extended position.

4. The ureteroscope system of claim 3, wherein the distal portion of the catheter includes a coiled spring housed at least partially therein and having a distal end region secured to the one or more wires or cables such that the distal portion is held in the retracted position with the coiled spring held in tension when the base is in the first position and moving the base to the second position at least partially releases tension in the coiled spring to extend the distal portion to the extended position.

5. The ureteroscope system of claim 2, wherein the actuator includes a motor positioned in the interior region of the handpiece and operably connected to the distal portion and configured to extend and retract the distal portion of the catheter relative to the proximal portion of the catheter.

6. The ureteroscope system of claim 5, further comprising one or more wires or cables secured to the motor and the distal portion of the catheter, wherein the motor is configured to increase an amount of the one or more wires or cables between the motor and the distal portion of the catheter to extend the distal portion to the extended position and configured to decrease an amount of the one or more wires or cables between the motor and the distal portion of the catheter to retract the distal portion to the retracted position.

7. The ureteroscope system of claim 6, wherein the distal portion of the catheter includes a coiled spring housed at least partially therein and having a distal end region secured to the one or more wires or cables such that increasing the amount of the one or more wires or cables between the motor and the distal portion of the catheter extends the distal portion to the extended position and decreasing the amount of the one or more wires or cables between the motor and the distal portion of the catheter retracts the distal portion to the retracted position.

8. The ureteroscope system of claim 2, wherein the actuator includes a pneumatic or hydraulic pump positioned in the interior region of the housing and configured to:
pneumatically or hydraulically increase a pressure in at least a portion of the catheter effective to deform at least the distal portion of the catheter to extend the distal portion of the catheter to the extended position; and
pneumatically or hydraulically decrease a pressure in at least a portion of the catheter effective to deform at least the distal portion of the catheter to retract the distal portion of the catheter to the retracted position.

9. The ureteroscope system of any of claims 2-8, wherein the distal portion telescopes at least partially into the catheter in the retracted position.

10. The ureteroscope system of any of claims 2-9, wherein the control member includes a selectively slidable tab on the handpiece.

11. The ureteroscope system of any of claims 2-9, wherein the control member includes an electronic device separate from the handpiece.

12. The ureteroscope system of any of claims 1-11, further comprising:
wherein the proximal portion of the catheter includes an active bend portion distal to the handpiece and proximate to the distal portion of the catheter;
a wire wheel secured to the interior region of the handpiece;
a steering controller on the handpiece connected to the wire wheel and configured to at least partially rotate the wire wheel in two different directions;
one or more wires secured to the active bend portion of the catheter and adjustable responsive to rotation of the wire wheel effective to at least partially bend the active bend portion.

13. The ureteroscope system of any of claims 1-12, wherein the distal portion is configured to extend about 2 mm to about 30 mm in the extended position relative to the retracted position of the distal portion.

14. The ureteroscope system of any of claims 1-13, further comprising a hydrophilic coating on at least the distal portion of the catheter.

15. A method of operating a catheter of a ureteroscope in a subject, the method comprising:
inserting the catheter of the ureteroscope into a urethra of a subject to a selected position; and
selectively extending a distal portion of the catheter from a retracted position to an extended position using a control member.

16. The method of claim 15, wherein selectively extending a distal portion of the catheter from a retracted position to an extended position using a control member includes activating, with the control member, an actuator disposed in the interior region of the handpiece and operably connected to the distal portion to extend the distal portion of the catheter relative to the proximal portion of the catheter.

17. The method of claim 16, wherein activating, with the control member, an actuator disposed in the interior region of the handpiece and operably connected to the distal portion to extend the distal portion of the catheter relative to the proximal portion of the catheter includes moving a base of the actuator positioned in the interior region of the handpiece from a first position to a second position to at least partially release tension on one or more wires or cables secured to the base and the distal portion of the catheter.

18. The method of claim 17, wherein the distal portion of the catheter includes a coiled spring housed at least partially therein and having a distal end region secured to the one or more wires or cables such that moving the base of the actuator from the first position to the second position at least partially releases tension on one or more wires or cables and the coiled spring in the distal portion.

19. The method of claim 16, wherein activating, with the control member, an actuator disposed in the interior region of the handpiece and operably connected to the distal portion to extend the distal portion of the catheter relative to the proximal portion of the catheter includes activating a motor positioned in the interior region of the handpiece to increase an amount of one or more wires or cables between the motor and the distal portion of the catheter to extend the distal portion to the extended position.

20. The method of claim 16, wherein activating, with the control member, an actuator disposed in the interior region of the handpiece and operably connected to the distal portion to extend the distal portion of the catheter relative to the proximal portion of the catheter includes activating a pneumatic or hydraulic pump positioned in the interior region of the housing to pneumatically or hydraulically increase a pressure in at least a portion of the catheter and deform at least the distal portion of the catheter to extend the distal portion of the catheter to the extended position.

21. The method of any of claims 15-20, wherein the distal portion telescopes out from the catheter when the distal portion is selectively extended.

22. The method of any of claims 15-21, wherein the control member includes a selectively slidable tab on the handpiece.

23. The method of any of claims 15-21, wherein the control member includes an electronic device separate from the handpiece.

24. The method of any of claims 15-23, further comprising bending an active bend portion of the catheter before selectively extending the distal portion of the catheter.

25. The method of any of claims 15-24, wherein selectively extending the distal portion of the catheter includes selectively extending the distal portion of the catheter about 2 mm to about 30 mm.

26. The method of any of claims 15-25, wherein at least a distal portion of the catheter includes a hydrophilic coating on at least the distal portion of the catheter.
